(19) United States
(12) Patent Application Publication
(10) Pub. No.: US 2025/0073238 A1
(43) Pub. Date: Mar. 6, 2025

(54) METHODS OF TREATING SYSTEMIC LUPUS ERYTHEMATOSUS USING BTK INHIBITORS

(71) Applicant: BeiGene Switzerland GmbH, Basel (CH)

(72) Inventors: Nan HU, Beijing (CN); Shuran LI, Beijing (CN); Xiaomin SONG, Beijing (CN); Zhen YAO, Beijing (CN); Yunhang Guo, Beijing (CN); Zhiwei WANG, Beijing (CN)

(21) Appl. No.: 18/253,720

(22) PCT Filed: Nov. 19, 2021
(86) PCT No.: PCT/CN2021/131725

§ 371 Date: May 19, 2023

(30) Foreign Application Priority Data
Nov. 20, 2020 (WO) PCT/CN2020/130482

(51) Int. Cl. A61K 31/519; A61P 37/02

(52) U.S. Cl. CPC A61K 31/519; A61P 37/02

(57) ABSTRACT

Provided herein are methods of treating systemic lupus erythematosus including lupus nephritis, in particularly active proliferative lupus nephritis in a subject with (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof.

### FIELD OF THE DISCLOSURE

Disclosed herein are methods of treating systemic lupus erythematosus including lupus nephritis, in particularly active proliferative lupus nephritis in a subject, comprising administering to the subject in need thereof (S)-7-(1-acryloylpiperidin-4-yl)-2-(4-phenoxyphenyl)-4,5,6,7-tetrahydropyrazolo-[1,5-a]pyrimidine-3-carboxamide or a pharmaceutically acceptable salt thereof.

### BACKGROUND OF THE DISCLOSURE

Systemic lupus erythematosus (SLE) is a complex, chronic autoimmune disease of unknown etiology that can affect almost any organ system, which follows a relapsing and remitting disease course. SLE occurs much more often in women than in men, up to 9 times more frequently in some studies, and often appears during the child-bearing years (from 15 to 45 years of age). In SLE, the immune system attacks the body's cells and tissues, resulting in inflammation and tissue damage which can harm the heart, joints, skin, lungs, blood vessels, liver, kidneys, and nervous system.

Lupus nephritis is a common and severe manifestation of SLE. Lupus nephritis is a type of glomerulonephritis characterized by the accumulation of immune complexes in glomeruli and often an inflammatory response in all kidney compartments. Over time, inflammation leads to chronic damage of the renal parenchyma and loss of kidney function. The incidence and prevalence of lupus nephritis are influenced by age, gender, race, ethnicity, geographic region, and diagnostic criteria. Clinically evident lupus nephritis is found in 20% to 60% of patients with SLE, and it occurs most often within 6 months of the SLE diagnosis. The standardized mortality ratio in SLE patients is 2- to 5-fold greater than in the general population (Bernatsky S, Boivin J-F, Joseph L, et al. Mortality in systemic lupus erythematosus. Arthritis Rheum. 2006; 54(8):2550-7.), and it increases further in patients who develop chronic kidney disease and end-stage renal disease (ESRD) (Mok C C, Kwok R C L, Yip P S F, et al. Effect of renal disease on the standardized mortality ratio and life expectancy of patients with systemic lupus erythematosus. Arthritis Rheum. 2013; 65(8):2154-60.).

Renal biopsy is the gold standard to diagnose lupus nephritis, and the treatment of lupus nephritis should be guided by the pathological classification. According to International Society of Nephrology/Renal Pathology Society Classification of Lupus Nephritis, lupus nephritis is classified into 6 classes. Patients with Class I and II lupus nephritis are usually treated as dictated by the extrarenal clinical manifestations of lupus. Class III lupus nephritis (focal lupus nephritis) and Class IV lupus nephritis, due to a worse prognosis, should be treated with corticosteroids and immunosuppressive agents. Initial therapy with corticosteroids combined with either cyclophosphamide or mycophenolate mofetil (MMF) lasts for 6 months. After initial therapy is completed, patients with Class III and Class IV lupus nephritis will receive initial therapy is completed, patients with Class III and Class IV lupus nephritis will receive maintenance therapy with azathioprine or mycophenolate mofetil and low doses of oral corticosteroids (≤10 mg/day prednisone or equivalent). After a complete remission is achieved, maintenance therapy should be continued for at least 1 year before the immunosuppressants are tapered off (KDIGO Clinical Practice Guideline for Glomerulonephritis. Kidney Int Suppl. 2012; 2(2):259-74.). However, data from several clinical studies have suggested that after 6 months of induction therapy, less than 30% of patients achieved a complete renal response.

To date, only voclosporin is approved for the treatment of lupus nephritis in the United States, and mycophenolate mofetil is approved in China, but the complete remission rate remains low. So there is still a large unmet need for new alternative treatments that can provide significant benefit for patients without incurring a high safety risk.

### SUMMARY OF THE DISCLOSURE

WO2014/173289A disclosed a series of BTK inhibitors, particularly (S)-7-(1-acryloylpiperidin-4-yl)-2-(4-phenoxyphenyl)-4,5,6,7-tetra-hydropyrazolo[1,5-a]pyrimidine-3-carboxamide (hereinafter Compound 1). Compound 1 can be used for the treatment of cancers with aberrations in the B-cell receptor (BCR) and FcR signaling pathway in which BTK plays important roles and has been demonstrated to have potent and irreversible inhibitory activities against BTK.

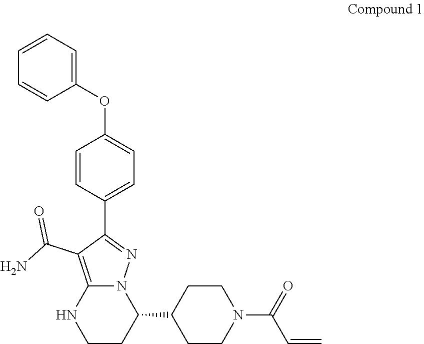 [SMILES: C=CC(=O)N1CCC([C@@H]2CCNc3c(C(N)=O)c(-c4ccc(Oc5ccccc5)cc4)nn32)CC1]

Compound 1 is a potent, specific, and irreversible BTK inhibitor with a favorable pharmacologic and pharmacokinetic (PK) profile. Compound 1 has good selectivity, against off-target kinases, including epidermal growth factor receptor (EGFR), Janus Kinase 3 (JAK3), human epidermal growth factor receptor-2 (HER2), TEC, inducible T-cell kinase (ITK), and others based on results from kinase inhibition and cell-based assays. The good selectivity of Compound 1 for BTK may result in a lower incidence and less severe off-target toxicities linked to inhibition of the aforementioned kinases.

The present disclosure describes (S)-7-(1-acryloylpiperidin-4-yl)-2-(4-phenoxyphenyl)-4,5,6,7-tetra-hydropyrazolo[1,5-a]pyrimidine-3-carboxamide (Compound 1) or a pharmaceutically acceptable salt thereof, showed a response in subjects with systemic lupus erythematosus (SLE).

The present disclosure also describes (S)-7-(1-acryloylpiperidin-4-yl)-2-(4-phenoxyphenyl)-4,5,6,7-tetra-hydropyrazolo[1,5-a]pyrimidine-3-carboxamide (Compound 1) or a pharmaceutically acceptable salt thereof, showed a response in subjects with lupus nephritis.

The present disclosure also describes (S)-7-(1-acryloylpiperidin-4-yl)-2-(4-phenoxyphenyl)-4,5,6,7-tetra-hydropyrazolo[1,5-a]pyrimidine-3-carboxamide (Compound 1) or a pharmaceutically acceptable salt thereof, showed a response in subjects with active proliferative lupus nephritis.

The inventor of the present disclosure discovered that Compound 1 showed efficacy in NZBWF1/J lupus mouse model, especially, when it was orally dosed at 3 and 10 mg/kg BID, Compound 1 showed a stronger anti-lupus effect as compared with mycophenolate mofetil (MMF).

The inventors of the present disclosure also discovered that Compound 1 demonstrated efficacy in systemic lupus erythematosus like chronic graft-versus-host-disease (SLE-cGVHD) mouse model. Twice daily oral dosing of Compound 1 at 10 and 20 mg/kg inhibited the level of anti-dsDNA IgG in serum, significantly reduced the proteinuria level and ameliorated splenomegaly.

In addition, the inventors of the present disclosure discovered that Compound 1 also showed dose-dependent efficacy in the range of 1.5 mg/kg to 50 mg/kg twice a day in the MRL/lpr mouse lupus model.

Compound 1 exposure of 40 mg twice a day (BID) in humans was close to that of 15 mg/kg twice a day in MRL/lpr mice. Furtherly, the efficacious dose of a BTK inhibitor should achieve sustained BTK occupancy >90% in PBMCs and >70% in spleens. Compound 1 at a dose from 80 mg per day (40 mg BID) to 320 mg per day (160 mg BID) was recommended, according to PK-PD simulation based on the preclinical data, PK variability, and the clinical dose in hematologic indications, which has been shown to be efficacious and well-tolerated in patients with B cell malignancies.

Compound 1 could be an effective therapy in treating systemic lupus erythematosus including lupus nephritis by reducing the proteinuria and increasing the remission rate significantly compared to the current standard therapy. Compound 1 could be well tolerated in systemic lupus erythematosus including lupus nephritis patients as chronic therapy.

### BRIEF DESCRIPTION OF THE DRAWINGS

FIG. 1A-a and FIG. 1A-b showed Compound 1 dose-dependently suppressed proteinuria. As a positive control, mycophenolate mofetil (MMF) was used once daily at 100 mg/kg; Compound 1 at 0.1 and 0.3 mg/kg was less effective than MMF whereas Compound 1 at 1, 3 and 10 mg/kg showed no significant difference when compared with MMF. (p<0.05; ** p<0.01, *** p<0.001 vs. Vehicle by Proportional Odds Model; MMF: Mycophenolate mofetil)

FIGS. 1A-c and 1A-d showed Compound 1 significantly reduced BUN level and inhibited kidney pathological injury. Compound 1 at 3 and 10 mg/kg and MMF remarkably inhibited BUN level. Compound 1 inhibited renal histopathology score in a dose-dependent manner, while MMF was not able to significantly inhibit the histopathology score. (* p<0.05 vs. Vehicle by one-way ANOVA; BUN: Blood urea nitrogen)

FIG. 1A-e showed Compound 1 significantly ameliorated splenomegaly. Compound 1 at 3 and 10 mg/kg dramatically inhibited spleen weight, while MMF was not able to significantly inhibit the spleen mass. (* p<0.05 vs. Vehicle by one-way ANOVA; MMF: Mycophenolate mofetil)

FIG. 1A-f and FIG. 1A-g showed Compound 1 significantly reduced TNFα and IL-10 levels in serum. Compound 1 at 10 mg/kg dramatically inhibited secretion of TNFα and IL-10. In contrast, MMF showed lower levels of TNFα and IL-10 with no statistical significance compared with the control group. (* p<0.05 vs. Vehicle by one-way ANOVA; MMF: Mycophenolate mofetil)

FIG. 1B-a showed the effect of Compound 1 on anti-dsDNA IgG level in the SLE-cGvHd mouse model. On 15 h day treatment, the level of anti-dsDNA IgG in serum was detected by ELISA. Data was presented as mean anti-DNA±standard error of the mean (SEM) in each group. Statistical analysis was conducted using One-way ANOVA (followed by Dunnett's test). ** p<0.01 vs. Vehicle.

FIG. 1B-b showed the effect of Compound 1 on proteinuria level Compound 1 in SLE-cGvHD mouse model. On the 22^{nd} day treatment, proteinuria level was assessed by determination of urine albumin using URIT 1 vp strips. Data was presented as mean score±standard error of the mean (SEM) of in each group. Statistical analysis was conducted using One-way ANOVA (followed by Dunnett's test). * p<0.05, ** p<0.01, **** p<0.0001 vs. Vehicle.

FIG. 1B-c showed the effect of Compound 1 on spleen-index in SLE-cGvHD mouse model. Spleen and body weight were measured at the end of the study. Spleen index (ratio of spleen weight to body) was calculated. Data was presented as mean spleen index±standard error of the mean (SEM) of in each group. Statistical analysis was conducted using One-way ANOVA (followed by Dunnett's test). ** p<0.01, *** p<0.001 vs. Vehicle.

FIG. 1B-d showed the effect of Compound 1 on body weight in SLE-cGvHD mouse model. Body weight was measured twice per week. Data are presented as mean body weight was measured twice per week. Data was presented as mean body weight x±standard error of the mean (SEM) of in each group.

FIG. 1C-a showed the effect of Compound 1 on anti-dsDNA IgG in MRL/MpJ-Fas^{lpr}/J (MRL/lpr) mouse model. At the end of the study, the level of anti-dsDNA IgG in serum was detected by ELISA. Data were presented as mean anti-dsDNA±standard error of the mean (SEM) in each group. Statistical analysis was conducted using One-way ANOVA (followed by Dunnett's test). ** p<0.01 vs. Vehicle.

FIG. 1C-b showed the effect of Compound 1 on proteinuria level in MRL/MpJ-Fas^{lpr}/J (MRL/lpr) mouse model. On the 118^{th} day, proteinuria level was assessed by determination of urine albumin using URTI 1 vp strips. Data were presented as mean score±standard error of the mean (SEM) in each group. Statistical analysis was conducted using One-way ANOVA (followed by Dunnett's test). **** p<0.0001 vs. Vehicle.

FIG. 1C-c showed the effect of Compound 1 on spleen-index in MRI/MpJ-Fas^{lpr}/J (MRI/lpr) mouse model. Spleen and body weight were measured at the end of the study.

Spleen index (ratio of spleen weight to body weight) was calculated. Data was presented as mean spleen index±standard error of the mean (SEM) of in each group. Statistical analysis was conducted using One-way ANOVA (followed by Dunnett's test). **** p<0.0001 vs. Vehicle; ##p<0.01 vs. Vehicle, ####p<0.0001 vs. Prednisone.

FIG. 1C-d showed the effect of Compound 1 on body weight in MRI/MpJ-Fas^{lpr}/J (MRL/lpr) mouse model. Body weight was measured twice per week. Data was presented as mean body weight±standard error of the mean (SEM) of in each group.

FIG. 1C-e showed the effect of Compound 1 on BUN level in MRL/MpJ-Fas^{lpr}/J (MRL/lpr) mouse model. Serum blood urea nitrogen (BUN) was measured at the end of the study. Data was present as mean BUN±standard error of the mean (SEM) in each group. Statistical analysis was conducted using One-way ANOVA (followed by Dunnett's test). ** p<0.01 vs. Vehicle.

FIG. 1C-f showed the effect of Compound 1 on kidney histopathology in MRI/MpJ-Fas^{lpr}/J (MRL/lpr) mouse model. Mice were euthanized using carbon dioxide at the end of the study. The kidneys were collected, a death score of inflammatory cell infiltration, glomerulopathy and cast were quantitatively analyzed. Data was presented as mean score±standard error of the mean (SEM) in each group. Statistical analysis was conducted using One-way ANOVA (followed by Dunnett's test). ** p<0.01, *** p<0.001, **** p<0.0001, vs. Vehicle.

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “therapeutically effective amount” as herein used, refers to the amount of a Bcl-2 inhibitor that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition. In some embodiment of present disclosure, the subject is a human.

The present disclosure provides a method of treating lupus nephritis in a subject, comprising administering to the subject in need thereof Compound 1 or a pharmaceutically acceptable salt thereof.

### Methods of Treatment

In one aspect, the present disclosure provides a method of treating systemic lupus erythematosus (SLE). SLE refers to a chronic, inflammatory, variable autoimmune disease of connective tissue that occurs chiefly in women and is typically characterized by fever, skin rash, fatigue, and joint pain and often by disorders of the blood, kidneys, heart, lungs, and brain (such as hemolytic anemia, nephritis, pleurisy, pericarditis, cognitive dysfunction, or meningitis). According to the main affected organs or tissues, SLE can be further classified into diseases including but not limited to, lupus nephritis, neuropsychiatric lupus, lupus pneumonia, lupus myocarditis and lupus hepatitis.

In one aspect, the present disclosure provides a method of treating lupus nephritis in a subject.

Lupus nephritis refers to glomerulonephritis associated with systemic lupus erythematosus that is typically characterized by proteinuria and hematuria and that often leads to renal failure. Lupus nephritis is classified into 6 histopathological classes. See Table 1.

In certain aspects, the method comprises administering to the subject in need thereof Compound 1 or a pharmaceutically acceptable salt thereof.

Lupus nephritis is classified into 6 histopathological classes. See Table 1.

TABLE 1
Abbreviated International Society of Nephrology/Renal Pathology
Society Classification of Lupus Nephritis^{a} (2003)

| | |
|---|---|
| Class I | Minimal mesangial lupus nephritis |
| Class II | Mesangial proliferative lupus nephritis |
| Class III | Focal lupus nephritis^{b} |
| | Type A (active lesions), Type A/C (active and |
| | chronic lesions), and Type C (chronic lesions) |
| Class IV | Diffuse segmental (IV-S) or global (IV-G) lupus nephritis^{c} |
| | Type A (active lesions), Type A/C (active and |
| | chronic lesions), and Type C (chronic lesions) |
| Class V | Membranous lupus nephritis^{d} |
| Class VI | Advanced sclerosing lupus nephritis |

Source: The Classification of Glomerulonephritis in Systemic Lupus Erythematosus Revisited (Weening JJ, VD D'Agati, MM Schwartz, et al. The classification of glomerulonephritis in systemic lupus erythematosus revisited. JASN 2004; 15 (2) 241-50.).
^{a}Indicates the grades (mild, moderate, severe) of tubular atrophy, interstitial inflammation and fibrosis, and severity of arteriosclerosis or other vascular lesions.
^{b}Indicates the proportion of glomeruli with active lesions and with sclerotic lesions.
^{c}Indicates the proportion of glomeruli with fibrinoid necrosis and cellular crescents.
^{d}Class V may occur in combination with Class III or IV, in which case both will be diagnosed.

In some embodiment of the present disclosure, lupus nephritis is active proliferative lupus nephritis.

Active proliferative lupus nephritis refers to class III/IV lupus nephritis (LN), which is classified by the International Society of Nephrology/Renal Pathology Society Classification of Lupus Nephritisa (2003).

Compound 1 can be administered by any suitable means, including oral, parenteral, intrapulmonary, and intranasal, and, if desired for local treatment, intralesional administration. Dosing can be by any suitable route. Various dosing schedules including but not limited to single or multiple administrations over various time-points, bolus administration, and pulse infusion are contemplated herein.

Compound 1 would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some embodiments, Compound 1 is orally administrated at a dose of 10 mg-640 mg per day, preferably 10 mg-320 mg per day, more preferably 40-320 mg per day, most preferably 80-320 mg per day. In some embodiments, Compound 1 is orally administrated at a dose of 40 mg-160 mg per day, preferably 80 mg-160 mg per day.

In some embodiments, Compound 1 is orally administrated at a dose of 5 mg-320 mg twice daily (BID), preferably 5 mg-160 mg BID, more preferably 20 mg-160 mg BID, most preferably 40 mg-160 mg BID.

In some embodiments, Compound 1 is orally administrated at a dose of 10 mg-640 mg once daily (QD), preferably 10 mg-320 mg QD, more preferably 40 mg-320 mg QD, most preferably 40 mg-160 mg QD.

### EXAMPLES

The present invention is further exemplified, but not limited to, by the following examples that illustrate the invention.

### Example 1A Efficacy of Compound 1 in NZBWF1/J Lupus Model

### Method

90 NZBWF1 female mice at 26 weeks of age were randomized into 7 groups with 10 mice per group, based on proteinuria level, anti-dsDNA IgG level and body weight. Treatment was initiated after randomization once daily (QD) with 100 mg/kg Mycophenolate mofetil (MMF) and twice daily (BID) with vehicle (0.5% MC), 0.1, 0.3, 1, 3 and 10 mg/kg Compound 1 for 96 days. Treatments were administered by oral gavage (p.o.) in a volume of 10 ml/kg body weight.

Mice were also being monitored daily for clinical signs of toxicity for the duration of the study. Body weight for each animal was recorded twice per week and proteinuria was measured weekly. Proteinuria was assessed using URIT 1 vp strips, and was scored on a 0-4 scale with scored as follows: 0=trace, 1≥30 mg/dL, 2≥100 mg/dL, 3≥300 mg/dL, and 4≥500 mg/dL.

On the day 96^{th} of treatment, blood was collected quickly by the heart punctures under carbon dioxide euthanasia. The 300-500 μL blood was transferred to the coagulation tube, and the upper serum samples were separated by centrifuge at 2,000 g for 10 minutes, and then stored in the refrigerator at −80° C. for later use. 110 μL serum was used for the detection of blood urea nitrogen level, and 25 μL serum was used for measurement of TNFα and IL-10 levels by Luminex.

The kidney was removed and fixed with 10% neutral buffer formalin solution. The tissue was taken from the modified block, dehydrated with alcohol step by step, embedded in paraffin, sectioned by slide slicer (about 3 μm thick), stained with hematoxylin-cosin (HE), and examined by light microscopy for pathological changes of the kidney tissue. Sections were blindly scored as the sum of three systems, including inflammation (0-4), glomerular damage (0-4), and sclerosis (0-4).

### Result

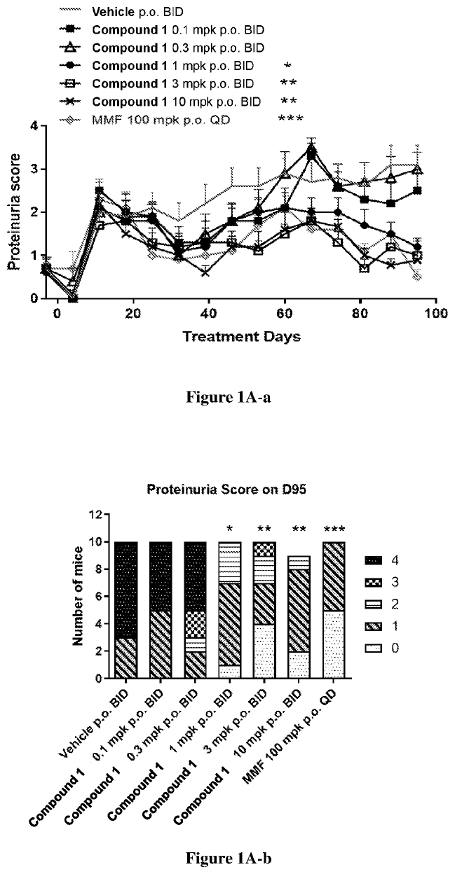

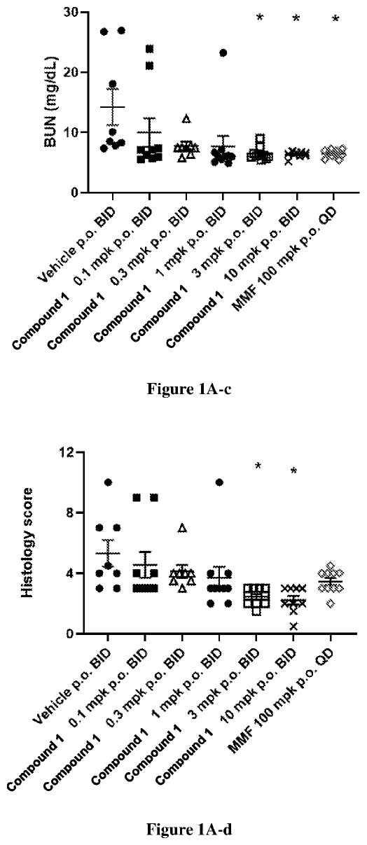

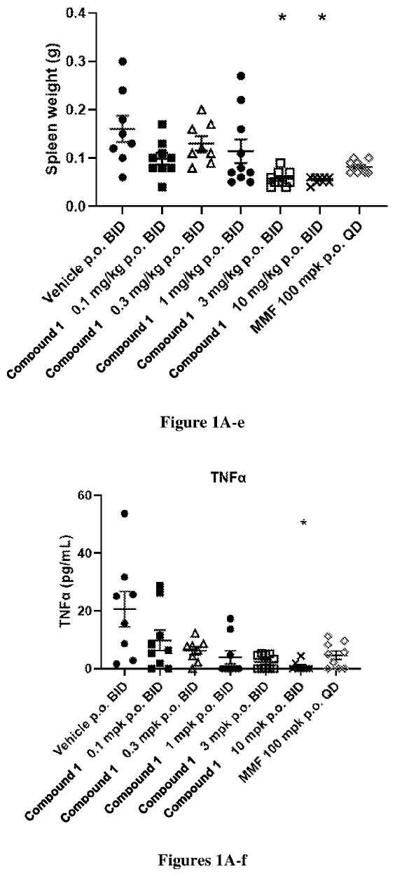

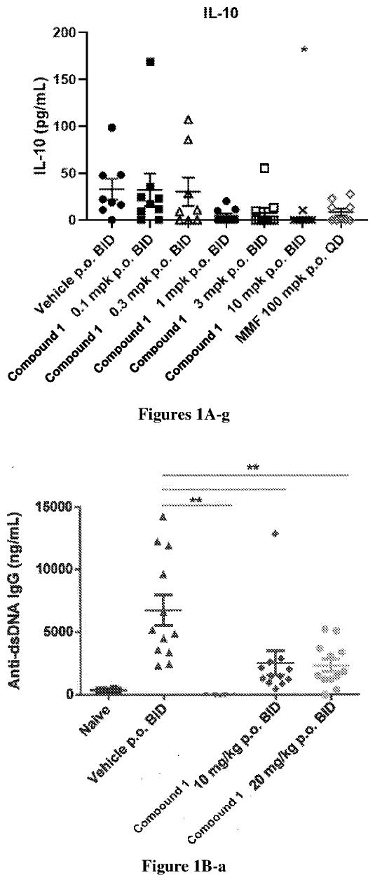

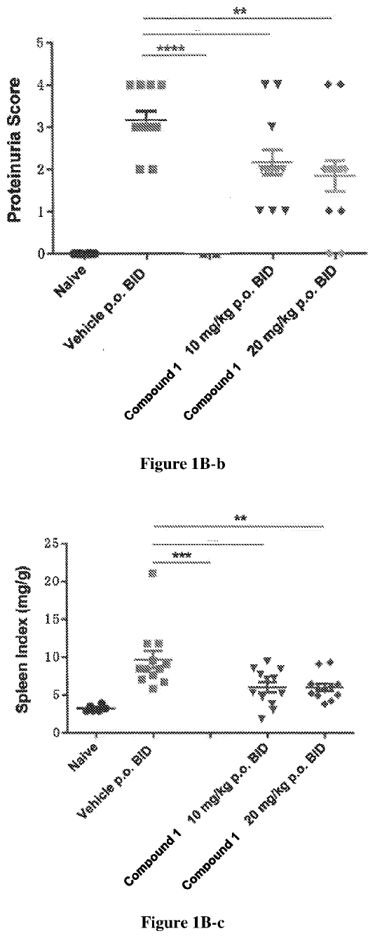

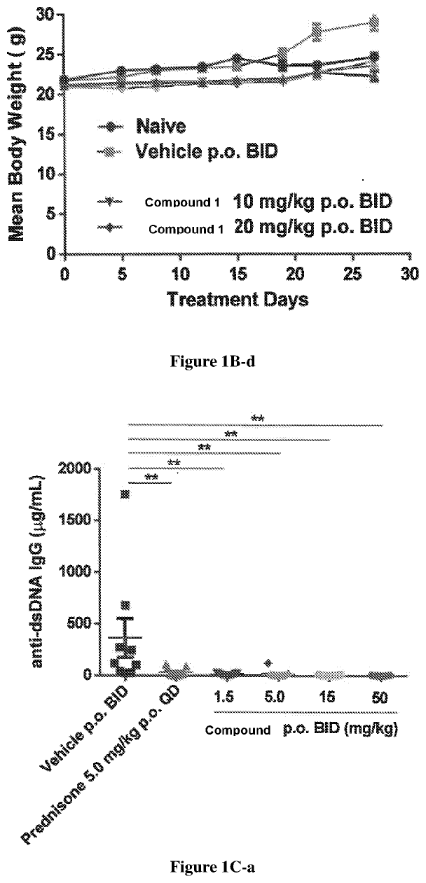

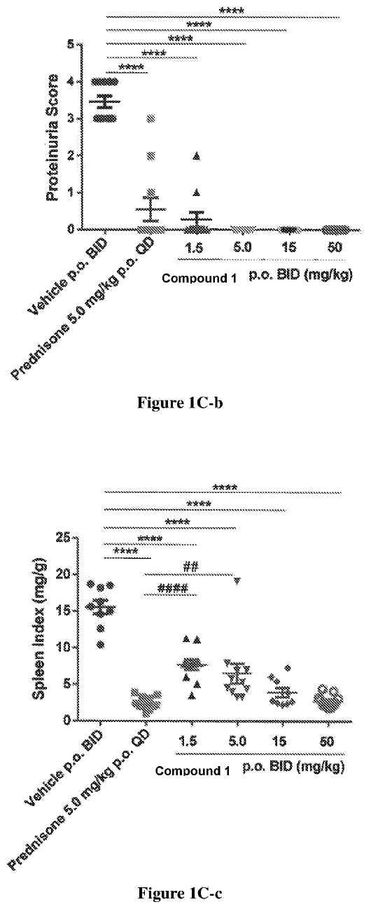

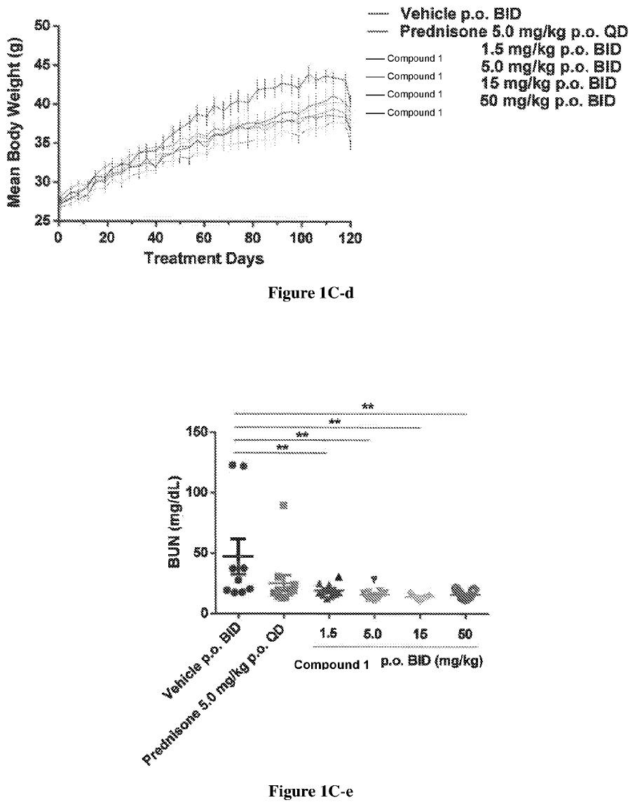

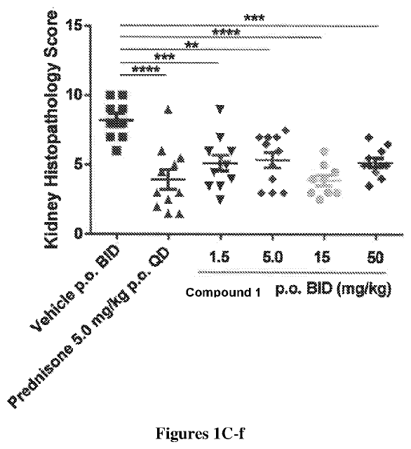

In vivo efficacy of Compound 1 was examined in NZBWF1/J lupus model, mycophenolate mofetil (MMF) was used once daily at 100 mg/kg as a positive control. Twice daily oral treatment with Compound 1 at 0.1 and 0.3 mg/kg was less effective than MMF, whereas Compound 1 at 1, 3, and 10 mg/kg showed no significant difference when compared with MMF (FIGS. 1A-a and 1A-b).

Compound 1 at 3 and 10 mg/kg and MMF remarkably inhibited blood urea nitrogen (BUN) level (FIG. 1A-c), and Compound 1 inhibited renal histopathology score in a dose-dependent manner, while MMF was not able to significantly inhibit the histopathology score (FIGS. 1A-d). Compound 1 at 3 and 10 mg/kg dramatically inhibited spleen weight, while MMF was not able to significantly inhibit the spleen mass (FIG. 1A-e). Compound 1 at 10 mg/kg dramatically inhibited secretion of TNFα and IL-10 (FIGS. 1A-f and 1A-g). In contrast, MMF showed a lower level of TNFα and IL-10 with no statistical significance compared with the control group (FIGS. 1A-f and 1A-g). All the results show that Compound 1 has a strong anti-lupus effect in NZBWF1/J lupus mouse model.

### Example 1B Efficacy of Compound 1 in Systemic Lupus Erythematosus Like Chronic Graft-Versus-Host-Disease (SLE-cGVHD) Model

### Method

Splenocytes from DBA/2 mice were transplanted into 54 B6D2F1 mice intravenously at day −7 and day 0. 48 animals were randomly divided into 4 groups of 12 animals each group according to the inoculation order. Treatment was initiated the next day after randomization until the end of the 26th day. Mice were orally administered twice daily to the vehicle (0.5% methylcellulose), 10 or 20 mg/kg Compound 1. Treatments were administered by oral gavage (p.o.) in a volume of 10 ml/kg body weight.

Mice were monitored daily for clinical signs of toxicity for the duration of the study. Body weight for each animal was recorded twice per week. On day 15^{th} of treatment, blood was collected from the orbital venous sinus under isoflurane/oxygen anesthesia and the serum samples were prepared. An anti-ds DNA IgG ELISA kit (Chondrex, Inc., Cat. 3031) was used to quantitate anti-ds DNA IgG levels in sera from individual mice.

On day 8^{th}, b 15^{th} and 22^{th} of treatment, proteinuria was assessed by determination of urine albumin using URIT 1 vp strips (URIT, China), and was scored on a 0-4 scale with scored as follows: 0=trace, 1≥30 mg/dL, 2≥100 mg/dL, 3≥300 mg/dL, and 4≥500 mg/dL.

On day 26^{th} of treatment, blood samples were collected from the retro-orbital sinus under isoflurane/oxygen anesthesia at 0, 0.5, 1.5, 4 and 8 hours for the Compound 1 treatment group. Plasma was collected by centrifugation at 5, 600 rpm for 7 minutes. Bioanalysis was conducted at 3D BioOptima Co., Ltd. (Suzhou, China).

The difference between the mean values of proteinuria level, spleen-index and anti-ds DNA IgG level in treatment and vehicle group was analyzed for significance using One-way ANOVA (followed by Dunnett's test). P<0.05 was considered statistically significant.

### Result

In vivo efficacy of Compound 1 was examined in systemic lupus erythematosus like chronic graft-versus-host-disease (SLE-cGVHD) mouse model. Twice daily oral dosing of Compound 1 10 and 20 mg/kg inhibited the level of anti-dsDNA IgG in serum, significantly reduced proteinuria level and ameliorated splenomegaly (FIGS. 1B-a, 1B-b and 1B-c). All treatment groups had no significant body weight throughout the study (FIG. 1B-d). Drug exposure of Compound 1 (AUC₀₋₈ₕ and Cₘₐₓ) at steady state increased dose dependently. All the results show Compound 1 ameliorated disease in SLE-cGvHD mouse model.

### Example 1C Efficacy of Compound 1 in MRL/Apr Lupus Model

### Method

MRL/MpJ-Fas^{lpr}/J (MRL/lpr) mice purchased from Jackson laboratories were randomized into 6 groups with 11 mice per group, based on proteinuria level, anti-dsDNA IgG and body weight. Treatment was initiated the next day after randomization once daily (QD) with 5.0 mg/kg prednisone or twice daily (BID) with vehicle (0.5% MC, methylcellulose), 1.5, 5, 15, and 50 mg/kg Compound 1 for 17 weeks. Treatments were administered by oral gavage (p.o.) in a volume of 10 ml/kg body weight.

During the experimental period, the condition and mortality of the mice were recorded every day. Body weight for each animal was recorded twice per week.

Proteinuria was measured by determination of urine albumin weekly using URIT 1 vp strips (URIT, China), and was scored on a 0-4 scale with scored as follows: 0=trace, 1≥30 mg/dLl, 2≥100 mg/dL, 3≥300 mg/dLl, and 4≥500 mg/dL.

Blood samples were collected from the retro-orbital sinus under isoflurane/oxygen anesthesia at 0, 0.5, 1.5, 4 and 8 hours for the Compound 1 treatment group. Plasma was collected by centrifugation at 5, 600 rpm for 7 minutes and was kept frozen at −80° C. until analysis. Bioanalysis was conducted at 3D BioOptima Co., Ltd. (Suzhou, China).

At the end of the study, blood was collected from the orbital venous sinus under isoflurane/oxygen anesthesia. The serum samples were prepared and analyzed for BUN level in serum. An anti-dsDNA IgG ELISA kit (Chondrex, Inc., Cat. 3031) was used to quantitate IgG level to anti-dsDNA IgG in sera from individual mice.

Mice were euthanized using carbon dioxide, spleen and body weight were measured at the end of the study. Spleen index (ratio of spleen weight to body weight) was calculated.

The kidneys were collected and preserved in 10% NBF, the tissues were trimmed, dehydrated, embedded in paraffin, sectioned (˜3 μm) and stained with HE. The histopathology exam was performed.

The difference between the mean values of proteinuria level, spleen-index, BUN level, kidney histopathology score and anti-dsDNA IgG level in treatment and vehicle group was analyzed for significance using One-way ANOVA (followed by Dunnett's test). P<0.05 was considered statistically significant.

### Result

In vivo efficacy of Compound 1 was examined in MRL/MpJ-Fas^{lpr}/J (MRI/lpr) model. Prednisone was used once daily at 5.0 mg/kg as a positive control. Twice daily oral treatment with Compound 1 at 1.5, 5, 15 and 50 mg/kg significantly protected mice from lupus nephritis, inhibited the anti-dsDNA IgG level in serum when compared with vehicle (FIG. 1C-a), significantly decreased proteinuria a serum blood urea nitrogen (BUN) level (FIGS. 1C-b and 1C-e), suppressed splenomegaly (FIG. 1C-c) with the dose-dependent manner and reduced the kidney histopathological score (FIGS. 1C-f). All the treatment groups had no significant impact on body weight throughout the study (FIG. 1C-d). Drug exposure of Compound 1 (AUC₀₋₈ₕ and Cₘₐₓ) at steady state increased dose independently. All the results show that Compound 1 has a strong anti-lupus effect in MRI/MpJ-Fas^{lpr}/J (MRI/lpr) mouse model.

### Example 2 a Phase 2, Multicenter, Randomized, Double-Blind, Placebo-Controlled Study to Evaluate the Safety and Efficacy of Compound 1 in Patients with Active Proliferative Lupus Nephritis

The primary objective of this study is to evaluate the efficacy of Compound 1 added to the standard of care as measured by the complete renal response for participants with active proliferative lupus nephritis.

### Study Design:

Patients will be randomized to one of the treatment groups according to the following arms and interventions.

| Arms | Assigned Interventions |
|---|---|
| Experimental: Compound 1 | Drug: Compound 1 |
| Participants will receive Compound 1 | Administered as specified |
| 40 mg twice daily (BID) for 72 weeks | in the treatment arm |
| Experimental: Compound 1 | Drug: Compound 1 |
| Participants will receive Compound 1 | Administered as specified |
| 160 mg twice daily (BID) for 72 weeks | in the treatment arm |
| Experimental: Compound 1 | Drug: Compound 1 |
| Participants will receive Compound 1 | Administered as specified |
| 160 mg once daily (QD) for 72 weeks | in the treatment arm |
| Experimental: Placebo | Drug: Placebo |
| Participants will receive placebo | Placebo matching |
| matching to Compound 1 for 72 weeks | to Compound 1 |

### Outcome Measures

### Primary Outcome Measure:

1. Proportion of participants with complete renal response [Time Frame: Week 49 Day 1]

### Secondary Outcome Measure:

1. Proportion of participants achieving complete renal response [Time Frame: Week 25 Day 1]
2. Proportion of participants achieving partial renal response [Time Frame: Week 25 Day 1 and Week 49 Day 1]
3. Proportion of participants achieving overall renal response [Time Frame: Week 25 Day 1 and Week 49 Day 1]
4. Time to first complete renal response: The time from the date of randomization to the date of the first complete renal response [Time Frame: Until Week 73 Day 1]
5. Time to first partial renal response: The time from the date of randomization to the date of the first partial renal response [Time Frame: Until Week 73 Day 1]
6. Change in total Systemic Lupus Erythematosus Disease Activity Index 2000 (SLEDAI-2K) score [Time Frame: Week 25 Day 1 and Week 49 Day 1]
SLEDAI-2K score is a systemic lupus erythematosus (SLE) activity index based on the presence of 24 features in 9 organ systems which ranges from 0-105, with higher scores indicating more severe features in participants in the past 30 days.
8. Pharmacokinetics of Compound 1 in patients with lupus nephritis, including Area under plasma concentration time curve (AUC) [Time Frame: Week 1 Day 1 and Week 5 Day 1]

### Eligibility

### Key Inclusion Criteria:

1. 18 to 70 years of age (inclusive) on the day of signing the Informed Consent Form (ICF), female or male
2. Clinical diagnosis of SLE according to the Systemic Lupus International Collaborating Clinics 2012 criteria.
3. ISN/RPS Class III/IV lupus nephritis [Type III(A), III (A+C), IV (A), and IV (A+C)], with or without Class V, as confirmed by a renal biopsy.
4. Positive antinuclear antibodies, positive anti-dsDNA autoantibody, and/or positive anti Smith autoantibody at screening.
5. Has 24-hour urine protein excretion >1.0 g at screening.

### Key Exclusion Criteria:

Exclusion criteria related to systemic lupus erythematous and other diseases:
1. Glomerulonephritis caused by reasons other than systemic lupus erythematous.
2. Sclerosis in >50% of glomeruli on renal biopsy.
3. Any other inflammatory diseases that might confound the assessments of efficacy, including but not limited to rheumatoid arthritis, myositis, vasculitis, or overlapping syndrome.
4. Severe extrarenal SLE, including but not limited to pulmonary arterial hypertension, severe myocarditis, severe central nervous system lupus (such as neuropsychiatric SLE, seizures, psychosis, transverse myelitis, central nervous system vasculitis and optic neuritis), etc.

Compound 1 could be an effective therapy in treating lupus nephritis by reducing the proteinuria and increasing the remission rate significantly compared to the current standard therapy. Compound 1 could be well tolerated in lupus nephritis patients as a chronic therapy.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

1. A method for treating systemic lupus erythematosus, the method comprising administering to a subject a therapeutically effective amount of (S)-7-(1-acryloylpiperidin-4-yl)-2-(4-phenoxyphenyl)-4,5,6,7-tetrahydropyrazolo-[1,5-a]pyrimidine-3-carboxamide (Compound 1) or a pharmaceutically acceptable salt thereof.

2. A method for treating lupus nephritis, the method comprising administering to a subject a therapeutically effective amount of (S)-7-(1-acryloylpiperidin-4-yl)-2-(4-phenoxyphenyl)-4,5,6,7-tetrahydropyrazolo-[1,5-a]pyrimidine-3-carboxamide (Compound 1) or a pharmaceutically acceptable salt thereof,

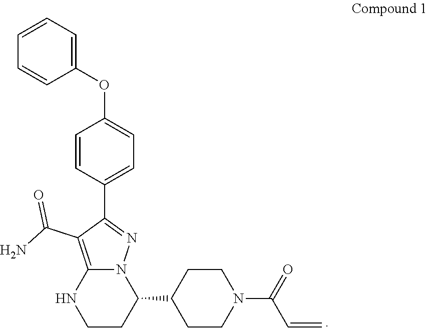 [SMILES: C=CC(=O)N1CCC([C@@H]2CCNc3c(C(N)=O)c(-c4ccc(Oc5ccccc5)cc4)nn32)CC1]

3. The method of claim 1, wherein the lupus nephritis is active proliferative lupus nephritis.

4. The method of any one of claims 1 to 3, wherein the subject is a patient, who meet one of the inclusion criteria consisting of the following, Clinical diagnosis of SLE according to the Systemic Lupus International Collaborating Clinics 2012 criteria;
a) ISN/RPS Class III/IV lupus nephritis [Type III (A), III (A+C), IV (A), and IV (A+C)], with or without Class V, as confirmed by a renal biopsy;
b) Positive antinuclear antibodies, positive anti-dsDNA autoantibody, and/or positive anti-Smith autoantibody at screening; and,
c) Having 24-hour urine protein excretion >1.0 g at screening.

5. The method of any one claims 1 to 3, wherein Compound 1 is administrated at a dose which achieve sustained BTK occupancy >90% in PBMCs and >70% in spleens.

6. The method of any one of claims 1 to 3, wherein Compound 1 is administered at a dose of 10 mg-640 mg per day, preferably 10 mg-320 mg per day, more preferably 40-320 mg per day, most preferably 80-320 mg per day.

7. The method of any one of claims 1 to 3, wherein Compound 1 is administered at a dose of 40 mg-160 mg per day, preferably 80 mg-160 mg per day.

8. The method of any one of claims 1 to 3, wherein Compound 1 is orally administrated at a dose of 5 mg-320 mg twice daily (BID), preferably 5 mg-160 mg BID, more preferably 20 mg-160 mg BID, most preferably 40 mg-160 mg BID.

9. The method of any one of claims 1 to 3, wherein Compound 1 is orally administrated at a dose of 10 mg-640 mg once daily (QD), preferably 10 mg-320 mg QD, more preferably 40 mg-320 mg QD, most preferably 40 mg-160 mg QD.

10. The method of any one of claims 1-8, wherein the subject shows an positive level of autoantibody including anti-dsDNA IgG in serum.

11. The method of claim 1, wherein systemic lupus erythematosus includes lupus nephritis, neuropsychiatric lupus, lupus pneumonia, lupus myocarditis and lupus hepatitis.

12. The method of claim 2, wherein lupus nephritis is any one of the following lupus nephritis:
Minimal mesangial lupus nephritis;
Mesangial proliferative lupus nephritis;
Focal lupus nephritis;
Diffuse segmental (IV-S) or global (IV-G) lupus nephritis;
Membranous lupus nephritis; or
Advanced sclerosing lupus nephritis.
